(19) United States
(12) Patent Application Publication
(10) Pub. No.: US 2026/0090728 A1
(43) Pub. Date: Apr. 2, 2026

(54) DEVICE FOR MEASURING BLOOD PRESSURE USING AN ADAPTIVE CUFF POSITIONING ASSEMBLY AND METHOD THEREOF

(71) Applicant: Aarogya Tech Inc., Reading, PA (US)

(72) Inventors: Amit Kumar Gupta, Bangalore (IN); Prashant Trivedi, Reading, PA (US)

(21) Appl. No.: 19/344,628

(22) Filed: Sep. 30, 2025

Related U.S. Application Data
(60) Provisional application No. 63/700,842, filed on Sep. 30, 2024.

(51) Int. Cl. A61B 5/021; A61B 5/022; A61B 5/06

(52) U.S. Cl. CPC A61B 5/02141; A61B 5/02225; A61B 5/065

(57) ABSTRACT

A device and method for measuring blood pressure using an adaptive cuff positioning assembly is disclosed. The device includes a resting fixture with embedded sensors to detect posture of a user, an armrest for supporting the adaptive cuff positioning assembly, and a linear actuation assembly to provide longitudinal motion. An enclosure coupled through an elevation actuator and a pivot assembly provides vertical and angular adjustment. The adaptive cuff positioning assembly comprises a cuff support structure for the upper arm, a displacement sensor for generating distance data, and a dual-bladder cuff assembly with a fitment bladder and a measurement bladder. The fitment bladder inflates to secure the cuff, while the measurement bladder inflates and deflates according to a pressure profile to obtain oscillometric signals. A control unit processes posture and distance data to actuate the assemblies and regulate inflation and deflation, enabling determination of systolic, diastolic, and mean arterial pressures.

### CROSS REFERENCE TO RELATED APPLICATION(S)

This application claims the priority to and incorporates by reference the entire disclosure of U.S. provisional patent application bearing No. 63/700,842 filed on Sep. 30, 2024

### TECHNICAL FIELD

Embodiments of the present disclosure relate to medical devices and more particularly relates to a device for measuring blood pressure of a user using an adaptive cuff positioning assembly without manual intervention.

### BACKGROUND

Blood pressure measurement is a vital diagnostic procedure in clinical and home healthcare environments. Conventional non-invasive blood b pressure measurement devices generally utilize a cuff wrapped around an upper arm of a user, which is manually secured before measurement. The non-invasive blood pressure measurement devices often employ a single inflatable bladder for both fitment and measurement and are typically dependent on manual placement and tightening of the cuff by the user or a healthcare provider.

Manual placement of the cuff introduces variability in the alignment of the cuff relative to a brachial artery and a heart level of the user. Improper positioning of the cuff may result in measurement errors, reduced repeatability, and discomfort to the user. Inconsistent placement further complicates long-term monitoring, particularly for the users with limited mobility or for applications where unattended operation is desirable.

Existing blood pressure measurement devices also provide limited or no automated adjustment of the cuff position relative to the arm of the user. While certain systems may allow minor mechanical adjustments, they generally lack integration of sensor-driven assemblies to detect posture data and to automatically align the cuff in longitudinal, vertical, or angular directions. As a result, such blood pressure measurement devices fail to consistently achieve correct anatomical positioning of the cuff, thereby reducing the accuracy of measured blood pressure values.

Furthermore, conventional cuffs typically employ a single bladder system, which performs both the fitment function and the measurement function. This arrangement often compromises measurement stability, as the cuff may slip or shift during inflation and deflation, particularly when used repeatedly by different users. Additionally, most blood pressure measurement devices do not incorporate sufficient fail-safe mechanisms within their pneumatic subsystems to address risks of overinflation, power failure, or pressure leakage.

Therefore, there is a need for an improved device for measuring blood pressure that reduces user-to-user variability, ensures consistent positioning of the cuff relative to the arm and heart level, and enhances safety and reliability of non-invasive blood pressure measurement.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a device for measuring blood pressure using an adaptive cuff positioning assembly is disclosed.

In one aspect, the device comprises a resting fixture, at least one armrest, at least one linear actuation assembly, an enclosure, the adaptive cuff positioning assembly, and a control unit. In another aspect, the resting fixture is configured with one or more embedded sensors to detect posture of a user for generating posture data to actuate the adaptive cuff positioning assembly proximate to an upper arm of the user. The one or more embedded sensors comprise at least one of: a load cell, a proximity sensor, and an optical sensor. The load cell is configured to determine weight of the user on the resting fixture for generating the posture data. The proximity sensor is configured to determine distance between the adaptive cuff positioning assembly and an arm of the user for generating the posture data. The optical sensor is configured to detect the posture of the user for determining a heart level of the user for generating the posture data. In other aspect, the at least one armrest operatively coupled to the resting fixture, which is configured to hold the adaptive cuff positioning assembly alongside the resting fixture.

Yet another aspect, the at least one linear actuation assembly is operatively coupled on the at least one armrest. The at least one linear actuation assembly is configured to provide a longitudinal linear motion to the adaptive cuff positioning assembly alongside the arm of the user based on the posture data. The longitudinal linear motion provided by the at least one linear actuation assembly is configured to translate the adaptive cuff positioning assembly forward and backward relative to the arm of the user. The at least one linear actuation assembly comprising one of: a mechanical actuation unit, a hydraulic actuation unit, and a pneumatic actuation unit, operatively coupled to one of: sliding rails, a guided linkage, rack and pinion, linear bearings and rails, a lead screw, and a ball screw.

In another aspect, the enclosure is pivotally coupled to the at least one linear actuation assembly using an elevation actuator and a pivot assembly. The enclosure is configured to provide support to a forearm of the user. The elevation actuator is configured to provide an elevation to the adaptive cuff positioning assembly to alter a vertical position for reaching to the heart level of the user based on at least one of: predefined anatomical reference data, the posture data, and stored user data. The elevation actuator is configured as one of: a telescopic actuator, a scissor lift actuator, a hydraulic cylinder, a pneumatic cylinder, a screw-driven actuator, and a linear motor, to provide the elevation to the adaptive cuff positioning assembly. The pivot assembly is configured to provide an angular motion to the adaptive cuff positioning assembly to align with a natural resting angle of the arm. The pivot assembly is configured as one of: a ball joint, a pin joint, and a hinge joint, to provide the angular motion to the adaptive cuff positioning assembly.

Yet another aspect, the adaptive cuff positioning assembly operatively coupled to the enclosure. The adaptive cuff positioning assembly comprises a contoured cradle, a cuff support structure, a displacement sensor, and a dual-bladder cuff assembly. The contoured cradle is operatively positioned on the enclosure, which is configured to provide support to an elbow of the user at a time of measuring the blood pressure. The cuff support structure is operatively connected to the contoured cradle, which is configured to provide support to the upper arm at the time of measuring the blood pressure. The displacement sensor is operatively positioned on the cuff support structure and is configured to generate distance data used to position the adaptive cuff positioning assembly at a predetermined location relative to the arm of the user. The displacement sensor comprises at least one of: an ultrasonic sensor, an optical sensor, and a capacitive sensor, to determine a predetermined distance of at least one inch above an elbow crease of the user.

The dual-bladder cuff assembly is operatively positioned inside the cuff support structure. The dual-bladder cuff assembly comprises a fitment bladder, and a measurement bladder. The fitment bladder and the measurement bladder are independently connected to a pneumatic subsystem through distinct fluid conduits. The pneumatic subsystem comprises one or more diaphragm pumps, one or more solenoid valves, and one or more fail-safe vents, to control inflation and deflation of the fitment bladder and the measurement bladder. The fitment bladder is configured to inflate by the pneumatic subsystem based on the distance data for gripping around the upper arm of the user. The measurement bladder is configured to inflate and deflate by the pneumatic subsystem according to a predetermined pressure profile for measuring the blood pressure. The measurement bladder is configured to inflate to a predetermined pressure at least 20 millimeters of mercury (mmHg) above a predictable systolic pressure of the user and to deflate according to a controlled deflation rate between 2 mmHg per second and 3 mmHg per second, defined by the control unit based on the predetermined pressure profile. The measurement bladder configured to deflate for obtaining oscillometric signals for determining systolic pressure, diastolic pressure, and mean arterial pressure of the user.

In other aspect, the control unit is operatively associated with the enclosure and is operatively connected to the one or more embedded sensors, the at least one linear actuation assembly, the elevation actuator, the displacement sensor, the dual-bladder cuff assembly, and one or more pressure sensors. The control unit is configured to process the posture data and the distance data to generate one or more actuation commands for: a) actuating the at least one linear actuation assembly in the longitudinal linear motion, b) triggering the elevation actuator to provide the elevation to the adaptive cuff positioning assembly, and c) controlling fluid communication within the pneumatic subsystem to inflate and deflate the fitment bladder and the measurement bladder, based on fitment bladder pressure data and measurement bladder pressure data obtained from the one or more pressure sensors, to measure the blood pressure. The control unit is configured to monitor the fitment bladder pressure data to verify optimal gripping of the upper arm of the user before initiating inflation of the measurement bladder. The control unit is further configured to trigger rapid deflation of the fitment bladder and the measurement bladder through the one or more fail-safe vents based on one of: the fitment bladder pressure data, the measurement bladder pressure data, and power failure conditions. The control unit configured to initiate measuring the blood pressure upon obtaining the posture data from the one or more embedded sensors.

In accordance with an embodiment of the present disclosure, a method for measuring blood pressure using the adaptive cuff positioning assembly is disclosed. In the first step, the method includes generating, by the one or more embedded sensors associated with the resting fixture, the posture data based on the posture of the user to actuate the adaptive cuff positioning assembly proximate to the upper arm of the user. In the next step, the method includes actuating, by the control unit, the at least one linear actuation assembly to provide the longitudinal linear motion to the adaptive cuff positioning assembly alongside the arm of the user based on the posture data.

In next step, the method includes triggering, by the control unit, the elevation actuator to provide the elevation to the adaptive cuff positioning assembly to alter the vertical position for reaching to the heart level of the user based on at least one of: the predefined anatomical reference data, the posture data, and the stored user data. In next step, the method includes providing, by the pivot assembly, the angular motion to the adaptive cuff positioning assembly to align with the natural resting angle of the arm. In next step, the method includes providing, by the contoured cradle associated with the adaptive cuff positioning assembly, support to the elbow of the user at the time of measuring the blood pressure

In next step, the method includes providing, by the cuff support structure associated with the adaptive cuff positioning assembly, support to the upper arm at the time of measuring the blood pressure. In next step, the method includes actuating, by the control unit, the at least one linear actuation assembly and the elevation actuator based on the distance data generated by the displacement sensor positioned on the cuff support structure to position the adaptive cuff positioning assembly at the predetermined location relative to the arm of the user.

In next step, the method includes controlling, by the control unit, the fluid communication within the pneumatic subsystem to inflate the fitment bladder associated with the dual-bladder cuff assembly based on the distance data and the fitment bladder pressure data obtained from the one or more pressure sensors to grip the upper arm of the user. In next step, the method includes controlling, by the control unit, the fluid communication within the pneumatic subsystem to inflate and deflate the measurement bladder associated with the dual-bladder cuff assembly according to the predetermined pressure profile for measuring the blood pressure.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limited in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1A illustrates an exemplary schematic view of a device for measuring blood pressure using an adaptive cuff positioning assembly, in accordance with an embodiment of the present disclosure;

FIG. 1B to FIG. 1D illustrates an exemplary detailed view of an adaptive cuff positioning assembly, in accordance with an embodiment of the present disclosure;

FIG. 1E illustrates an exemplary schematic view of a dual-bladder cuff assembly, in accordance with an embodiment of the present disclosure;

FIG. 1F illustrates an exemplary block diagram representation of the device, in accordance with an embodiment of the present disclosure;

FIGS. 2A and 2B illustrates an exemplary block diagram representation of the control unit, in accordance with an embodiment of the present disclosure;

FIGS. 2C and 2D illustrates an exemplary block diagram representation of a fitment bladder unit (FBU), in accordance with an embodiment of the present disclosure;

FIGS. 2E and 2F illustrates an exemplary block diagram representation of a measurement bladder unit (MBU), in accordance with an embodiment of the present disclosure; and

FIG. 3 illustrates an exemplary flow chart of a method for measuring blood pressure using the adaptive cuff positioning assembly, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

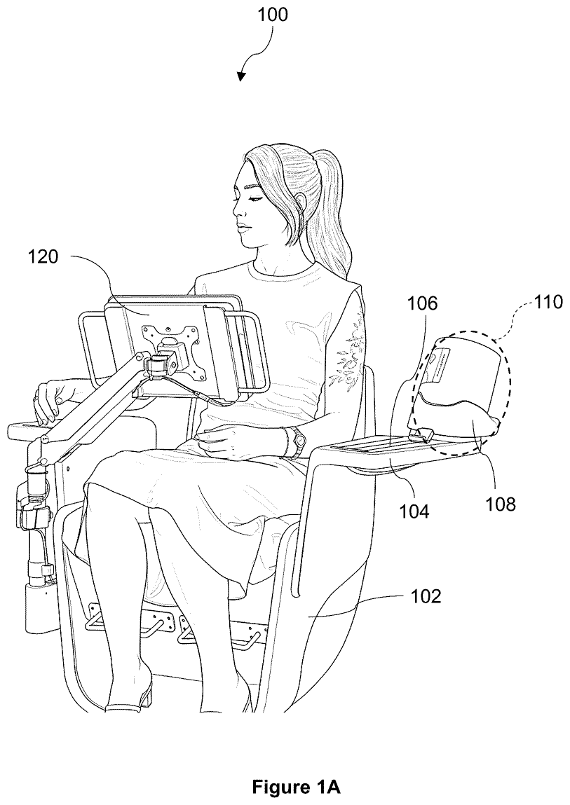

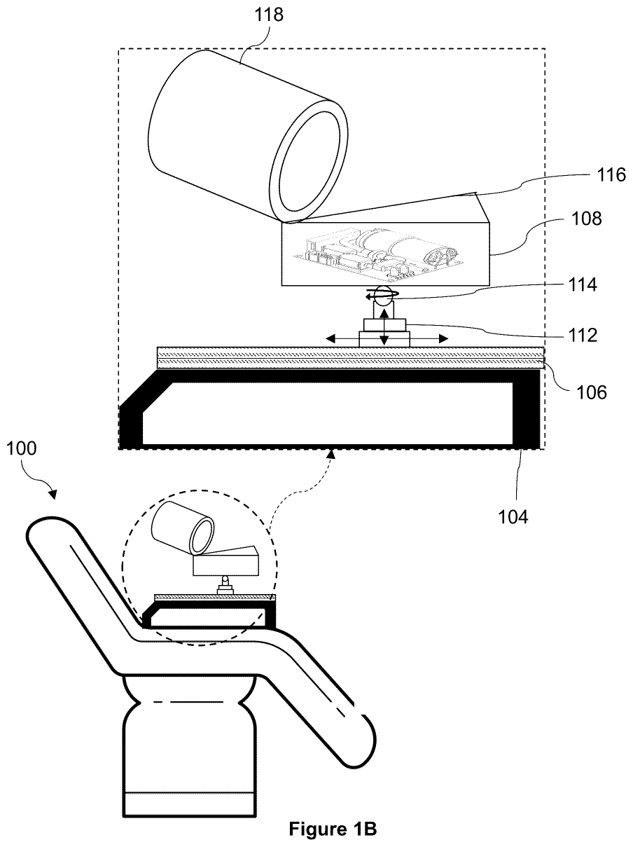

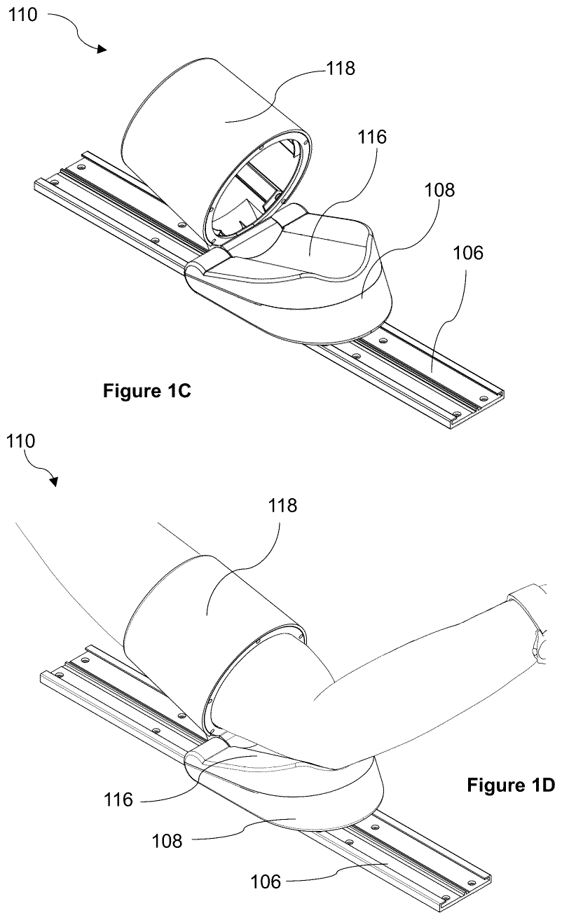

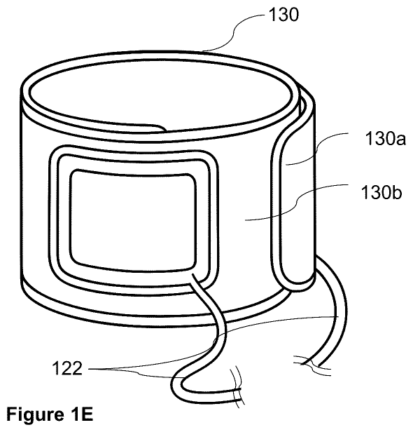

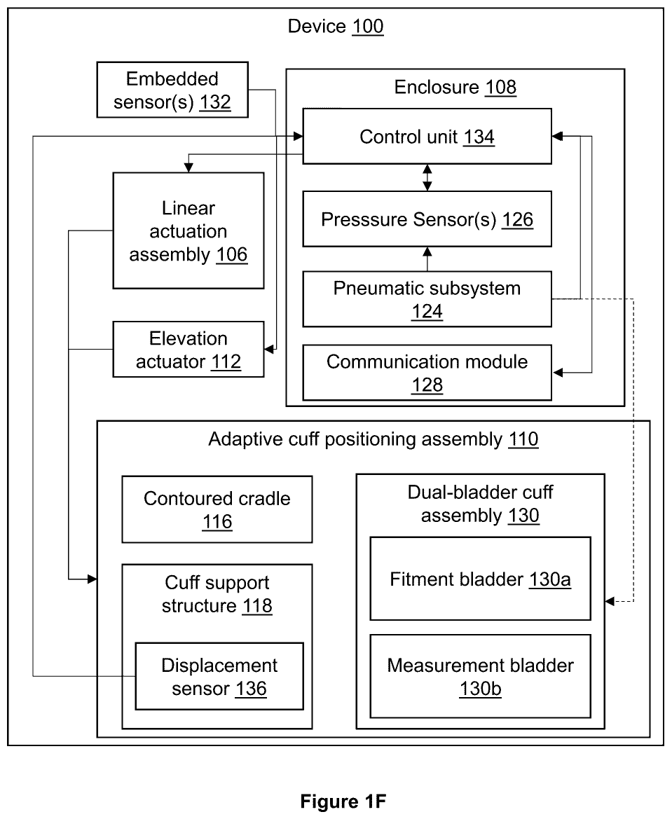

Referring now to the drawings, and more particularly to FIG. 1A through FIG. 3, where similar reference characters denote corresponding features consistently throughout the figures, there are shown preferred embodiments, and these embodiments are described in the context of the following exemplary system and/or method.

FIG. 1A illustrates an exemplary schematic view of a device 100 for measuring blood pressure using an adaptive cuff positioning assembly 110, in accordance with an embodiment of the present disclosure.

FIG. 1B to FIG. 1D illustrates an exemplary detailed view of an adaptive cuff positioning assembly 110, in accordance with an embodiment of the present disclosure.

FIG. 1E illustrates an exemplary schematic view of a dual-bladder cuff assembly 130, in accordance with an embodiment of the present disclosure.

FIG. 1F illustrates an exemplary block diagram representation of the device 100, in accordance with an embodiment of the present disclosure.

According to an exemplary embodiment of the present disclosure, the device 100 comprises a resting fixture 102, at least one armrest 104, at least one linear actuation assembly 106, an enclosure 108, the adaptive cuff positioning assembly 110, and a control unit 134. The device 100 is configured to automatically align and stabilize the adaptive cuff positioning assembly 110 relative to an upper arm of a user by processing posture and positional data obtained from the user. Unlike conventional devices that rely on manual placement and adjustment, the device 100 integrates multiple components to reduce user-to-user variability and improve measurement repeatability. The arrangement of the device 100 ensures ergonomic comfort, consistent anatomical alignment, and safe operation during the blood pressure measurement, thereby enhancing reliability of non-invasive monitoring while minimizing dependence on operator skill.

In an exemplary embodiment, the resting fixture 102 is configured with one or more embedded sensors 132 to detect posture of the user for generating the posture data to actuate the adaptive cuff positioning assembly 110 proximate to the upper arm of the user. The one or more embedded sensors 132 comprise at least one of: a load cell, a proximity sensor, and an optical sensor. In the illustrative embodiment, the resting fixture 102 may be configured as a seat, a bench, or a platform depending on the intended installation environment. In one example, the resting fixture 102 comprises a seat portion with an integrated back support, configured to stabilize the user in a comfortable and repeatable sitting position while enabling easy access of the upper arm to the adaptive cuff positioning assembly 110. In another example, the resting fixture 102 may be implemented as a tabletop platform where the user positions the arm for measurement, thereby eliminating the need for a full seating arrangement.

The load cell is configured to determine weight of the user on the resting fixture 102 for generating the posture data. For example, the load cell may be disposed beneath a seat portion or a support plate of the resting fixture 102, such that when the user sits or places an arm on the resting fixture 102, the load cell generates an electrical signal proportional to the weight or applied force. This signal is processed to confirm user presence and to establish a reference posture for subsequent positioning of the adaptive cuff positioning assembly 110.

The proximity sensor is configured to determine distance between the adaptive cuff positioning assembly 110 and an arm of the user for generating the posture data. In one example, the proximity sensor may be, but not limited to, one of: an infrared, an ultrasonic sensor, and the like, mounted within the resting fixture 102 or on the at least one armrest 104, which emits a signal toward the user's arm and receives a reflected signal. By calculating the time of flight or intensity of the reflected signal, the proximity sensor provides a distance measurement that enables the control unit 134 to estimate arm position and adjust the adaptive cuff positioning assembly 110 accordingly.

The optical sensor is configured to detect the posture of the user for determining a heart level of the user for generating the posture data. By way of example, the optical sensor may be, but not limited to, one of: a camera, an array of photodiodes, and the like, integrated into the resting fixture 102, directed toward an upper body of the user. The optical sensor may capture one of: image data and reflective light data that is processed using posture recognition model stored in the control unit 134. Based on this processing, the control unit 134 determines the relative vertical position of the user's upper arm with respect to an estimated heart level. The heart level reference is used as a calibration point to control the elevation actuator 112 and ensure the adaptive cuff positioning assembly 110 is positioned correctly.

In an exemplary embodiment, the posture recognition model is a computational method that uses computer vision or sensor data to automatically detect, analyze, and classify the position and orientation of a person's body or body parts, essentially identifying the user's posture. The posture recognition model may be implemented using one or more standard image-processing methods such as, but not limited to, at least one of: an edge detection model, a feature extraction model, and a geometric analysis model of image data captured by the optical sensor. In another example, the posture recognition model may be realized as a machine learning model, such as, but not limited to, one of: a convolutional neural network (CNN) and a statistical classifier, trained on datasets of annotated human body postures to identify anatomical landmarks such as a shoulder joint, the elbow crease, or the upper arm orientation. The training dataset may comprise image data from a plurality of individuals with varying body types, seating positions, and lighting conditions, thereby enabling the posture recognition model to generalize across different users.

The control unit 134 may execute the posture recognition model on the image data in real time using its processor and memory resources. For example, the control unit 134 may identify the vertical alignment of the shoulder joint relative to the seating position and use this as a reference point for calculating the estimated heart level. The control unit 134 then correlates the detected position of the upper arm with the heart level reference to generate the posture data. This posture data is subsequently used as an input to actuate the elevation actuator 112, which raises or lowers the adaptive cuff positioning assembly 110 to ensure that the cuff support structure 118 and the dual-bladder cuff assembly 130 are aligned with the heart level of the user.

In an exemplary embodiment, the at least one armrest 104 is operatively coupled to the resting fixture 102, which is configured to hold the adaptive cuff positioning assembly 110 alongside the resting fixture 102. The at least one armrest 104 provides a stable lateral support for the user's forearm and acts as a structural mounting point for the at least one linear actuation assembly 106. In one implementation, the at least one armrest 104 may be padded or contoured to enhance user comfort during prolonged use. The at least one armrest 104 may also integrate guide rails or housings through which the at least one linear actuation assembly 106 extends, thereby ensuring that the adaptive cuff positioning assembly 110 is able to translate smoothly alongside the resting fixture 102. By combining posture data obtained from the one or more embedded sensors 132 with a physical reference support provided by the at least one armrest 104, the device 100 ensures that the adaptive cuff positioning assembly 110 is consistently and reproducibly aligned with the user's upper arm for reliable blood pressure measurement.

In an exemplary embodiment, the at least one linear actuation assembly 106 is operatively coupled on the at least one armrest 104. The at least one linear actuation assembly 106 is configured to provide a longitudinal linear motion to the adaptive cuff positioning assembly 110 alongside the arm of the user based on the posture data. The longitudinal linear motion provided by the at least one linear actuation assembly 106 is configured to translate the adaptive cuff positioning assembly 110 forward and backward relative to the arm of the user. This translation ensures that the adaptive cuff positioning assembly 110 can be positioned proximate to the upper arm of users having different arm lengths, while also enabling fine adjustment for alignment with a brachial artery.

The at least one linear actuation assembly 106 comprising, but not limited to, one of: a mechanical actuation unit, a hydraulic actuation unit, a pneumatic actuation unit, and the like operatively coupled to one of: sliding rails, a guided linkage, rack and pinion, linear bearings and rails, a lead screw, a ball screw, and the like. In one example, the mechanical actuation unit may include an electric motor coupled to the lead screw or the ball screw, wherein the rotation of the screw causes a carriage connected to the adaptive cuff positioning assembly 110 to move forward or backward along the armrest 104. In another example, the hydraulic actuation unit may include a hydraulic cylinder integrated within the at least one of armrest 104, configured to extend or retract in response to pressurized fluid flow, thereby moving the adaptive cuff positioning assembly 110 in the longitudinal direction. Similarly, the pneumatic actuation unit may include a pneumatic cylinder or bellows device mounted within the armrest 104, wherein compressed air supplied through the pneumatic subsystem 124 provides linear extension or retraction to translate the adaptive cuff positioning assembly 110.

The coupling mechanisms for the at least one of linear actuation assembly 106 may include precision sliding rails or linear bearings and rails, which provide low-friction movement for smooth translation. In some embodiments, the guided linkage may be employed where multiple pivoted members are arranged to constrain the adaptive cuff positioning assembly 110 to move strictly along a forward-backward path relative to the arm of the user. A rack and pinion arrangement may also be used, wherein the rotation of a pinion gear driven by a motor enclosed in the enclosure 108 advances a linear rack element fixed to the adaptive cuff positioning assembly 110.

By way of example, in one implementation the linear actuation assembly 106 may consist of a motor-driven ball screw mounted beneath the armrest 104, with a carriage attached to the cuff support structure 118 of the adaptive cuff positioning assembly 110. When the motor is actuated by the control unit 134 based on posture data, the carriage moves smoothly along a length of the ball screw, translating the adaptive cuff positioning assembly 110 forward or backward to align precisely with the user's upper arm. This motor-driven ball screw arrangement allows for repeatable, controlled motion, minimizes backlash, and ensures that the adaptive cuff positioning assembly 110 can be accurately positioned even for users with different anthropometric dimensions.

The longitudinal linear motion enabled by the linear actuation assembly 106 provides not only ergonomic comfort for the user but also anatomical precision. By allowing the adaptive cuff positioning assembly 110 to travel toward or away from the user's torso, the device 100 reduces the risk of misalignment that may otherwise occur with a fixed-position cuff. As a result, the at least one linear actuation assembly 106 plays a critical role in ensuring that a cuff consistently achieves correct positioning for reliable and repeatable blood pressure measurement.

In an exemplary embodiment, the enclosure 108 is pivotally coupled to the at least one linear actuation assembly 106 using the elevation actuator 112 and a pivot assembly 114. The enclosure 108 serves as a structural housing that supports the adaptive cuff positioning assembly 110 and provides a surface for stabilizing the forearm of the user during measurement. In an exemplary embodiment, the enclosure 108 is configured to enclose the control unit 134, one or more microcontrollers, one or more low-dropout regulators (LDOs), a communication module 128, a fitment bladder unit (FBU), a measurement bladder unit (MBU), the one or more pressure sensors 126, the pneumatic subsystem 124, and the like. The enclosure 108 may be fabricated from a rigid material such as, but not limited to, one of: aluminum, stainless steel, reinforced polymer, and the like, and is dimensioned to withstand repetitive movement caused by actuation of the elevation actuator 112 and the pivot assembly 114 while ensuring comfort and stability for the user's arm. The enclosure 108 may further incorporate cushioning or padding at regions where the forearm makes contact to reduce user fatigue and to maintain the arm in a consistent orientation throughout the measurement cycle.

The elevation actuator 112 is configured to provide an elevation to the adaptive cuff positioning assembly 110 in order to alter its vertical position for reaching the heart level of the user. The elevation actuator 112 functions based on at least one of: predefined anatomical reference data, the posture data generated by the one or more embedded sensors 132, and stored user data. By combining these inputs, the elevation actuator 112 ensures that the cuff associated with the adaptive cuff positioning assembly 110 is raised or lowered to align with the user's heart level, thereby reducing hydrostatic pressure errors in the blood pressure measurement.

The elevation actuator 112 may be implemented as one of: a telescopic actuator, a scissor lift actuator, a hydraulic cylinder, a pneumatic cylinder, a screw-driven actuator, a linear motor, and the like. For example, in one implementation the elevation actuator 112 may include the telescopic actuator comprising nested tubular members driven by a motorized lead screw, such that extension or retraction of the elevation actuator 112 raises or lowers the enclosure 108. In another implementation, the scissor lift actuator driven by an electric motor may be used to expand or contract a crossed-linkage mechanism, thereby vertically displacing the enclosure 108 relative to the at least one of armrest 104. In a further example, the elevation actuator 112 may be realized as the hydraulic cylinder or the pneumatic cylinder mounted between the at least one linear actuation assembly 106 and the enclosure 108, wherein pressurized fluid or air is supplied through control valves to extend or retract a piston rod and adjust the height of the adaptive cuff positioning assembly 110. In yet another embodiment, the screw-driven actuator or the linear motor may be employed to provide highly precise vertical displacement with minimal mechanical play, allowing accurate positioning at the heart level of the user.

The pivot assembly 114 is configured to provide an angular motion to the adaptive cuff positioning assembly 110 to align with the natural resting angle of the arm of the user. The pivot assembly 114 is configured as one of, but not limited to, a ball joint, a pin joint, a hinge joint, and the like. For example, when implemented as the ball joint, the pivot assembly 114 enables multi-axis angular adjustment, allowing the enclosure 108 to tilt inward or outward to follow the neutral resting orientation of the arm. When configured as the pin joint or the hinge joint, the pivot assembly 114 provides rotation about a single axis, which is sufficient for aligning the adaptive cuff positioning assembly 110 with the lateral angle of the arm. In one implementation, the pivot assembly 114 may be coupled with a servo motor or stepper motor that provides controlled angular displacement under the command of the control unit 134. This allows the angular position of the adaptive cuff positioning assembly 110 to be adjusted automatically based on posture data, ensuring that the arm remains in a relaxed and ergonomic orientation during blood pressure measurement. By integrating the elevation actuator 112 and the pivot assembly 114 into the enclosure 108, the device 100 provides coordinated vertical and angular adjustment of the adaptive cuff positioning assembly 110. This ensures consistent anatomical alignment of the cuff relative to the heart level and the natural arm posture of the user, thereby reducing variability and improving the accuracy and repeatability of blood pressure measurements across a wide range of users.

In an exemplary embodiment, the adaptive cuff positioning assembly 110 is operatively coupled to the enclosure 108. The adaptive cuff positioning assembly 110 comprises a contoured cradle 116, a cuff support structure 118, a displacement sensor 136, and a dual-bladder cuff assembly 130. The contoured cradle 116 is operatively positioned on the enclosure 108, which is configured to provide support to an elbow of the user at a time of measuring the blood pressure. In one implementation, the contoured cradle 116 is formed with a recessed or cup-shaped geometry that conforms to the posterior aspect of the user's elbow joint, thereby preventing lateral or forward displacement of the arm during measurement. The contoured cradle 116 may be fabricated from rigid material with an over molded or padded surface to ensure comfort while maintaining positional stability. By locating the elbow at a fixed anatomical reference point, the contoured cradle 116 enables consistent and repeatable positioning of the upper arm relative to the cuff support structure 118.

The cuff support structure 118 is operatively connected to the contoured cradle 116 and is configured to provide support to the upper arm at the time of measuring the blood pressure. As illustrated in FIG. 1C, the cuff support structure 118 extends longitudinally from the contoured cradle 116, thereby ensuring that once the elbow is positioned within the contoured cradle 116, the upper arm is automatically aligned with the cuff support structure 118. The cuff support structure 118 incorporates as a cylindrical-shaped design with an open segment, which is dimensioned to receive and support the medial and lateral aspects of the upper arm. This cylindrical geometry allows the dual-bladder cuff assembly 130 to be circumferentially positioned around the arm while maintaining a stable and centered orientation relative to the brachial artery.

In another exemplary embodiment, the cuff support structure 118 may include adjustable features such as sliding panels, hinged segments, or modular inserts to vary the internal diameter of the cylindrical design. Such adjustability allows the cuff support structure 118 to accommodate a range of arm circumferences, for example from 22 centimeter (cm) to 42 cm, which corresponds to standard adult arm sizes used in clinical practice. The adjustability ensures that the dual-bladder cuff assembly 130 is consistently centered regardless of user anthropometry, thereby reducing measurement variability.

The outer body of the cuff support structure 118 may be fabricated from a rigid structural material such as, but not limited to, a medical-grade Acrylonitrile Butadiene Styrene (ABS) plastic, a polycarbonate, a lightweight aluminum alloy, and the like. The rigid structural material provide sufficient durability to withstand repeated actuation cycles from the at least one linear actuation assembly 106 and the elevation actuator 112, while maintaining dimensional stability of the cylindrical form to ensure reproducible positioning of the dual-bladder cuff assembly 130.

The displacement sensor 136 is operatively positioned on the cuff support structure 118 and is configured to generate distance data used to position the adaptive cuff positioning assembly 110 at a predetermined location relative to the arm of the user. The displacement sensor 136 comprises at least one of: an ultrasonic sensor, an optical sensor, and a capacitive sensor, to determine a predetermined distance of at least one inch above an elbow crease of the user. In one example, the displacement sensor 136 may be the ultrasonic transducer mounted within the cuff support structure 118, which emits pulses and measures the time of flight of reflected signals to determine the vertical distance between the cuff support structure 118 and the elbow crease. In another example, the displacement sensor 136 may include the optical sensor that capture images of the arm. The optical sensor may be configured as one of: a camera module, a line-scan sensor, and an array of photodiodes, integrated within the cuff support structure 118. The optical sensor captures image data or reflected light patterns from the surface of the upper arm, which are processed by the control unit 134 to determine the relative location of the elbow crease and the upper arm position within the adaptive cuff positioning assembly 110. Image-based analysis enables the control unit 134 to compute a reference distance corresponding to at least one inch above the elbow crease, which is used to guide actuation of the linear actuation assembly 106 and the elevation actuator 112. In one implementation, the optical sensor may employ infrared illumination to improve accuracy under varying ambient light conditions.

In another implementation, pattern-recognition model may be executed on the image data to reliably identify the anatomical landmarks of the user's arm. The pattern-recognition model is a system built using machine learning techniques such as, but not limited to, one of: neural networks and statistical methods, that is trained to automatically detect, identify, and classify recurring regularities, structures, or trends (patterns) in raw data associated with the elbow crease. The anatomical landmark of the elbow crease is selected as a reliable reference point because it represents a fixed anatomical transition between the forearm and upper arm, which allows consistent placement of the dual-bladder cuff assembly 130 at a predetermined distance above the crease. The distance data generated by the displacement sensor 136 is communicated to the control unit 134, which uses the distance data to actuate the linear actuation assembly 106 and the elevation actuator 112, thereby ensuring that the adaptive cuff positioning assembly 110 is positioned at the correct anatomical reference location above the elbow crease.

The pattern-recognition model may be implemented using a convolutional neural network (CNN) model, a support vector machine (SVM) model, a k-nearest neighbor classifier model, or a combination of such models, trained to process features extracted from the image data captured by the optical sensor integrated into the cuff support structure 118. Training data for the pattern-recognition model may comprise a plurality of images of human arms annotated with ground-truth elbow crease positions under varying lighting conditions, arm orientations, and skin tones. The training data may further include different anthropometric variations (arm lengths, circumferences, and body postures), thereby enabling the pattern-recognition model to generalize across different users.

The training process may include preprocessing steps such as image normalization, noise reduction, and augmentation (e.g., rotation, scaling, contrast adjustment) to improve robustness of the pattern-recognition model. The trained pattern-recognition model is then stored in memory associated with the control unit 134 and executed in real time to process live image data. In one embodiment, the control unit 134 may downsample or crop image regions around the elbow to reduce computational load and accelerate inference speed. In another embodiment, the pattern-recognition model may output a confidence score associated with detected landmarks, and only detections above a predefined threshold are used for actuation decisions.

Once the elbow crease is detected, the control unit 134 computes a reference position corresponding to at least one inch above the crease and generates actuation commands to the at least one linear actuation assembly 106 and the elevation actuator 112 to align the adaptive cuff positioning assembly 110. In some cases, the pattern-recognition model may be supplemented by other embedded sensors 132, such as a proximity sensor or a load cell, to perform sensor fusion and validate the positional accuracy.

In the context of the present disclosure, the CNN model refers to a machine learning model based on deep learning architectures that utilize convolutional layers, pooling layers, and fully connected layers to automatically extract spatial features and patterns from the image data, making it particularly effective for tasks such as detecting anatomical landmarks in optical sensor images of the arm. The SVM model refers to a supervised learning model that constructs hyperplanes in a high-dimensional feature space to separate data into distinct classes, enabling reliable classification of features such as edges or contours in images associated with the elbow crease. The k-NN model refers to a non-parametric model that classifies data points based on the majority label of the k closest data points in the feature space, which can be used to identify similarities in arm image features relative to previously labeled training examples.

In an exemplary embodiment, the dual-bladder cuff assembly 130 is operatively positioned inside the cuff support structure 118. The dual-bladder cuff assembly 130 comprises a fitment bladder 130a, and a measurement bladder 130b, each arranged to function independently while operating in coordination under the control of the control unit 134. The fitment bladder 130a and the measurement bladder 130b are independently connected to the pneumatic subsystem 124 through distinct fluid conduits, ensuring that inflation and deflation cycles of one bladder do not interfere with the function of the other.

The pneumatic subsystem 124 comprises one or more diaphragm pumps, one or more solenoid valves, and one or more fail-safe vents, operatively configured to regulate airflow into and out of the fitment bladder 130a and the measurement bladder 130b. In one example, the one or more diaphragm pumps may be fluidly connected to each bladder (the fitment bladder 130a and the measurement bladder 130b) through its own conduit and solenoid valve, enabling independent control of inflation pressure, deflation rate, and venting. The one or more fail-safe vents may be implemented as normally-open solenoid valves that are automatically triggered to vent the fitment bladder 130a and the measurement bladder 130b in the event of power loss or overpressure condition, thereby ensuring user safety in accordance with standard medical device safety protocols.

The fitment bladder 130a is configured to inflate by the pneumatic subsystem 124 based on the distance data generated by the displacement sensor 136. Upon receiving a positioning command from the control unit 134, the pneumatic subsystem 124 inflates the fitment bladder 130a until sufficient circumferential grip is achieved around the upper arm of the user. This grip stabilizes the arm within the cuff support structure 118 and ensures that the measurement bladder 130b is maintained in an anatomically correct orientation over the brachial artery. In one embodiment, the control unit 134 monitors the pressure data generated by a dedicated pressure sensor associated with the fitment bladder 130a to verify that an optimal gripping pressure has been achieved before initiating inflation of the measurement bladder 130b.

The measurement bladder 130b is configured to inflate and deflate by the pneumatic subsystem 124 according to a predetermined pressure profile for measuring the blood pressure. The measurement bladder 130b is configured to inflate to a predetermined pressure at least 20 millimeters of mercury (mmHg) above a predictable systolic pressure of the user. For example, if the user's systolic pressure is expected to be 120 mmHg based on historical stored data or initial trial inflation, the measurement bladder 130b inflates to at least 140 mmHg. This ensures complete occlusion of the brachial artery, which is required for oscillometric measurement.

Following inflation, the measurement bladder 130b is configured to deflate according to a controlled deflation rate between 2 mmHg per second and 3 mmHg per second, defined by the control unit 134 based on the predetermined pressure profile. This controlled deflation rate ensures sufficient temporal resolution of oscillometric pulses while preventing user discomfort. As the measurement bladder 130b deflates, the pneumatic subsystem 124 releases air through a solenoid valve under the control unit 134 regulation, and the one or more pressure sensors 126 associated with the measurement bladder 130b acquires the measurement bladder pressure data. The control unit 134 processes the measurement bladder pressure data to identify oscillometric signals corresponding to arterial pulsations.

The measurement bladder 130b is therefore configured to deflate in a controlled manner for obtaining the oscillometric signals that are analyzed to determine systolic pressure, diastolic pressure, and mean arterial pressure of the user. The systolic pressure refers to the maximum arterial pressure in the brachial artery during ventricular contraction of the heart (systole). The systolic pressure represents the peak pressure exerted against arterial walls when a left ventricle ejects blood into an aorta. Clinically, the systolic pressure is expressed in millimeters of mercury (mmHg) and is the higher of the two values reported in a blood pressure reading (e.g., the “120” in 120/80 mmHg). The diastolic pressure refers to the minimum arterial pressure in the brachial artery during ventricular relaxation of the heart (diastole), when heart chambers fill with blood. The diastolic pressure represents the baseline pressure maintained in an arterial system when no active cardiac contraction is occurring. Clinically, the diastolic pressure is expressed in millimeters of mercury (mmHg) and is the lower of the two values reported in a blood pressure reading (e.g., the “80” in 120/80 mmHg). The mean arterial pressure refers to the average arterial pressure in a single cardiac cycle, providing an indicator of overall tissue perfusion. The mean arterial pressure is not the arithmetic mean of systolic and diastolic values but is typically approximated using the formula:

$$MAP \approx Diastolic Pressure + \frac{1}{3} ( Systolic Pressure - Diastolic Pressure )$$

This accounts for the longer duration of diastole compared to systole in a normal cardiac cycle. The mean arterial pressure is clinically significant as it reflects the effective pressure driving blood through the systemic circulation. By integrating independent fluid pathways, closed-loop pressure control, and dual-bladder functionality within the cuff support structure 118, the dual-bladder cuff assembly 130 enables accurate, repeatable, and safe measurement of blood pressure while minimizing user-to-user variability.

In another exemplary embodiment, the fitment bladder 130a and the measurement bladder 130b are housed within the dual-bladder cuff assembly 130 and are arranged in a circumferentially aligned manner inside the cuff support structure 118. In one embodiment, the fitment bladder 130a and the measurement bladder 130b are formed of flexible, medical-grade polymeric materials such as, but not limited to, at least one of: thermoplastic polyurethane (TPU), silicone elastomer, and polyvinyl chloride (PVC) coated nylon. These medical-grade polymeric materials are selected for their biocompatibility, resistance to fatigue from repeated pressurization cycles, and ability to maintain airtight seals under pressure. The fitment bladder 130a and the measurement bladder 130b may have thicknesses in the range of 0.2 mm to 0.5 mm, sufficient to withstand inflation pressures up to 300 mmHg without rupture, while remaining compliant enough to conform to the contour of the upper arm.

The fitment bladder 130a may have a relatively larger contact surface and uniform geometry, for example an annular or toroidal chamber, to provide circumferential grip across a range of arm circumferences. The measurement bladder 130b, in contrast, may be shaped as an elongated chamber with a rectangular or elliptical footprint aligned longitudinally with the arm, thereby concentrating pressure over the region of the brachial artery. In one example, the fitment bladder 130a may occupy the outer layer of the dual-bladder cuff assembly 130, while the measurement bladder 130b is positioned immediately beneath it, closer to the arm surface. This layered configuration ensures that once the fitment bladder 130a is inflated to secure the cuff, the measurement bladder 130b can operate independently to generate the oscillometric signal.

The fitment bladder 130a and the measurement bladder 130b are fluidly connected to the pneumatic subsystem 124 via independent conduits, each reinforced with fluid conduits, quick-connect fittings, and one-way valves to prevent backflow. The fluid conduits are embedded within the cuff support structure 118, ensuring that airflow is directed precisely and that the fitment bladder 130a and the measurement bladder 130b can be inflated and deflated independently under the control of the control unit 134.

To enhance patient comfort, the fitment bladder 130a and the measurement bladder 130b may be encased in a fabric sleeve or laminated to a soft textile, such as polyester or cotton, which forms the user-facing surface of the cuff support structure 118. This textile covering provides breathability and prevents direct skin contact with the medical-grade polymeric materials. The dual-bladder cuff assembly configuration, combined with anatomical alignment over the elbow crease and brachial artery, ensures that blood pressure measurement is performed with high accuracy, reproducibility, and minimal user discomfort.

In an exemplary embodiment, the control unit 134 is operatively associated with the enclosure 108 and is operatively connected to the one or more embedded sensors 132, the at least one linear actuation assembly 106, the elevation actuator 112, the displacement sensor 136, the dual-bladder cuff assembly 130, and the one or more pressure sensors 126. The control unit 134 is implemented as a microcontroller-based system with a memory unit, a processor, and communication interfaces configured to execute a set of instructions for coordinating actuation and measurement functions of the device 100. In one example, the control unit 134 may be realized as a 32-bit microcontroller with integrated analog-to-digital converters (ADC) for processing sensor signals, digital-to-analog converters (DAC) or pulse-width modulation (PWM) outputs for actuator control, and a communication module 128 such as recommended standard 232, Inter-Integrated Circuit (I2C), or Serial Peripheral Interface (SPI) for interfacing with one or more end devices such as at least one of: a user interface 120, user communication devices, computing terminals, mobile devices, smartphones, Personal Digital Assistants (PDAs), tablet computers, phablet computers, wearable computing devices, Virtual Reality/Augmented Reality (VR/AR) devices, laptops, desktops, and the like.

In one embodiment, the user interface 120 is operatively connected to the control unit 134 and is configured to allow interaction between the user and the device 100 during operation. The user interface 120 may include one or more of: a touchscreen display, a graphical display with associated push buttons, a keypad, indicator lights, or an audio feedback system. In one example, the user interface 120 is realized as a touchscreen panel mounted on the enclosure 108, which displays measurement status, posture alignment guidance, and final blood pressure readings to the user. The touchscreen panel may further allow a user to initiate or terminate a measurement cycle, select preferences, or review stored data. In another example, the user interface 120 may include physical push buttons or capacitive touch keys that provide basic functions such as “Start,” “Stop,” or “Emergency Release,” with visual feedback provided through light-emitting diodes (LEDs). The LEDs may indicate operational states, such as cuff inflation in progress, measurement complete, or error conditions. The user interface 120 may also be configured with audio feedback capability, such as beeps or spoken prompts, to guide the user through the measurement process. For instance, an audio prompt may instruct the user to place their arm in the adaptive cuff positioning assembly 110 or remain still during measurement.

In one implementation, the user interface 120 is wirelessly connected to external devices via the communication module 128. Through the communication module 128, the measurement results may be transmitted to the one or more end devices for storage and further analysis. In such embodiments, the user interface 120 may be simplified to only essential indicators, while extended interaction occurs through the connected external device.

The control unit 134 is configured to process the posture data and the distance data to generate one or more actuation commands. Based on the posture data obtained from the one or more embedded sensors 132, the control unit 134 actuates the at least one linear actuation assembly 106 to perform longitudinal linear motion, thereby translating the adaptive cuff positioning assembly 110 forward or backward relative to the arm of the user. Similarly, based on a combination of the posture data, the predefined anatomical reference data, and the stored user data, the control unit 134 triggers the elevation actuator 112 to provide vertical adjustment to the adaptive cuff positioning assembly 110 for aligning the cuff centerline with the heart level of the user.

The one or more actuation commands comprise, but not limited to, at least one of: a command to actuate the at least one linear actuation assembly 106 to provide the longitudinal linear motion to the adaptive cuff positioning assembly 110 alongside the arm of the user; a command to trigger the elevation actuator 112 to alter the vertical position of the adaptive cuff positioning assembly 110 for aligning with the heart level of the user; a command to operate the pivot assembly 114 to adjust the angular orientation of the adaptive cuff positioning assembly 110 with respect to the natural resting angle of the arm; a command to control fluid communication within the pneumatic subsystem 124 for inflating or deflating the fitment bladder 130a and the measurement bladder 130b; a command to monitor pressure data generated by the one or more pressure sensors 126 for verifying optimal gripping or for detecting oscillometric signals; and a command to trigger rapid deflation of the fitment bladder 130a and the measurement bladder 130b through the one or more fail-safe vents upon detection of an abnormal condition.

The control unit 134 is further configured to control fluid communication within the pneumatic subsystem 124 to inflate and deflate the fitment bladder 130a and the measurement bladder 130b. The inflation and deflation cycles are regulated based on the fitment bladder pressure data and the measurement bladder pressure data obtained from the one or more pressure sensors 126. In one embodiment, the control unit 134 may modulate a pump driver front end associated with the one or more diaphragm pumps and actuate the one or more solenoid valves to control airflow within the pneumatic subsystem 124. This closed-loop control ensures precise pressurization of the fitment bladder 130a for gripping and accurate pressure ramps in the measurement bladder 130b for oscillometric signal acquisition.

The control unit 134 is configured to monitor the fitment bladder pressure data to verify optimal gripping of the upper arm of the user before initiating inflation of the measurement bladder 130b. For example, the control unit 134 may determine that the fitment bladder pressure has reached a predefined gripping threshold (e.g., 30-40 mmHg) and remains stable for a set duration before commanding inflation of the measurement bladder 130b. This determination step ensures that the adaptive cuff positioning assembly 110 is stabilized and prevents motion-related artifacts during the measurement cycle.

The control unit 134 is also configured to trigger rapid deflation of the fitment bladder 130a and the measurement bladder 130b through the one or more fail-safe vents. The one or more fail-safe vents are initiated based on one of: abnormal fitment bladder pressure data, abnormal measurement bladder pressure data, or detection of the power failure condition. In such cases, the one or more fail-safe vents, which may be normally-open the one or more solenoid valves, are automatically actuated to release air from the bladders, thereby preventing excessive cuff pressure on the user's arm and ensuring compliance with medical device safety standards.

In one embodiment, the control unit 134 is further configured to initiate measuring the blood pressure upon obtaining the posture data from the one or more embedded sensors 132. For instance, when the embedded sensors 132 detect that the user is correctly seated and the arm is positioned within the contoured cradle 116 and the cuff support structure 118, the control unit 134 automatically initiates the sequence of fitment bladder inflation, followed by measurement bladder 130b inflation and controlled deflation. This automation reduces dependence on operator input, minimizes user error, and ensures repeatability of blood pressure measurement across different users.

In one exemplary embodiment, the device 100 is detachably attached to the resting fixture 102. The detachable configuration allows the adaptive cuff positioning assembly 110 and associated components to be mounted or removed from the resting fixture 102 without requiring permanent installation. In another embodiment, the device 100 may be retrofitted onto existing resting fixtures, such as standard chairs, benches, or examination tables, by using modular mounting brackets, clamps, or fasteners. This retrofitting capability enables integration of the device 100 into various clinical or home environments without the need for specialized furniture. For example, the enclosure 108 and the at least one armrest 104 may be provided as an independent assembly that is secured onto the resting fixture 102 using quick-release mechanisms, thereby permitting portability, easy cleaning, or replacement. Such flexibility in attachment ensures broader applicability of the device 100 while maintaining consistent functionality of the adaptive cuff positioning assembly 110.

In alternative exemplary embodiment, the device 100 further comprises one or more additional sensors. The one or more additional sensors may also comprise, but not limited to, at least one of: one or more oscillometric sensors, one or more piezoelectric sensors, and the like. The one or more oscillometric sensors is configured to measure oscillations in an artery wall as the blood pressure cuff deflates. The oscillations are related to a pulse of the blood, which assists in detecting the systolic pressure and the diastolic pressure based on the changes in the oscillations. The one or more piezoelectric sensors is configured to detect changes in the pressure. The one or more piezoelectric sensors is sensitive to the vibrations caused by the blood flow and are used to identify systolic points and diastolic points.

In another exemplary embodiment, the device 100 is also actuated by manual actuation of a switch unit. The switch unit may comprise, but not constrained to, at least one of a: switch, button, and the like. The switch unit is one of: integrated into the device 100 and located at a convenient location for the one or more users. Upon actuation of the switch unit, the signal is transmitted to the control unit 134. The control unit 134 is configured to initiate the automated positioning of the adaptive cuff positioning assembly 110 for measuring the blood pressure.

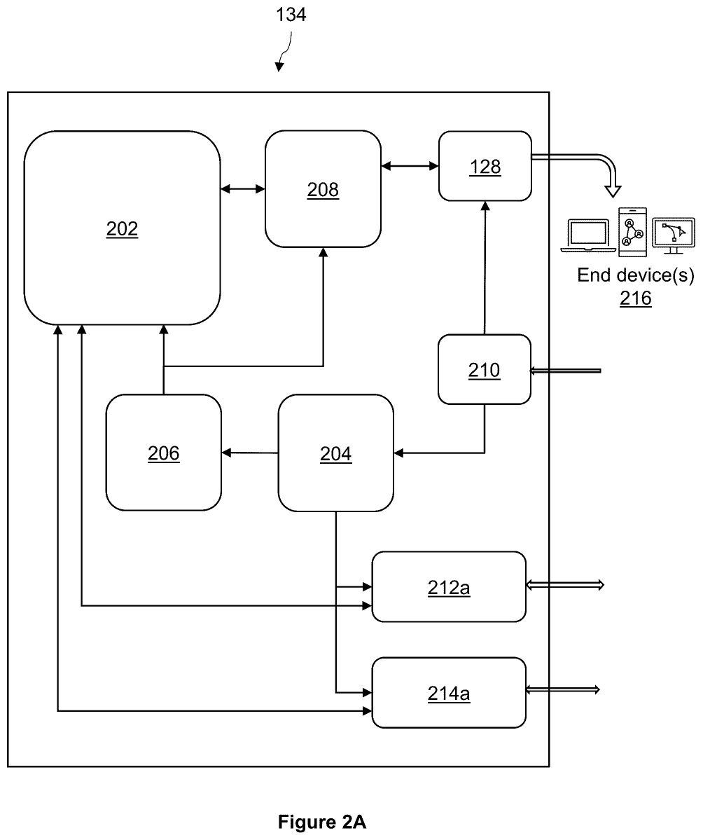

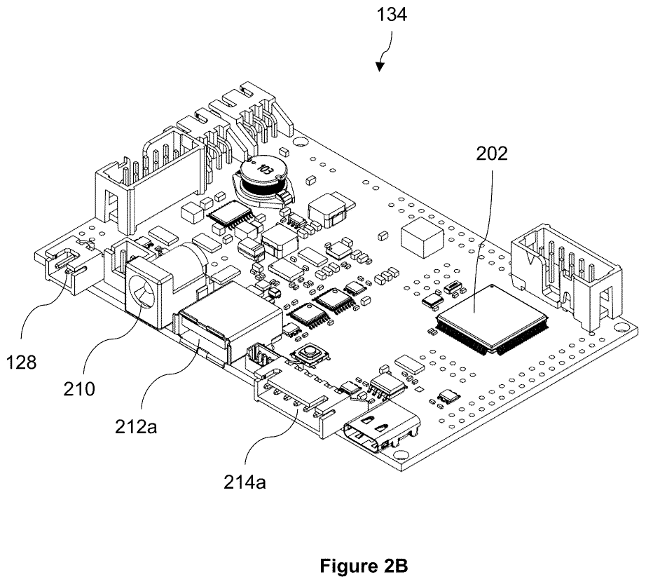

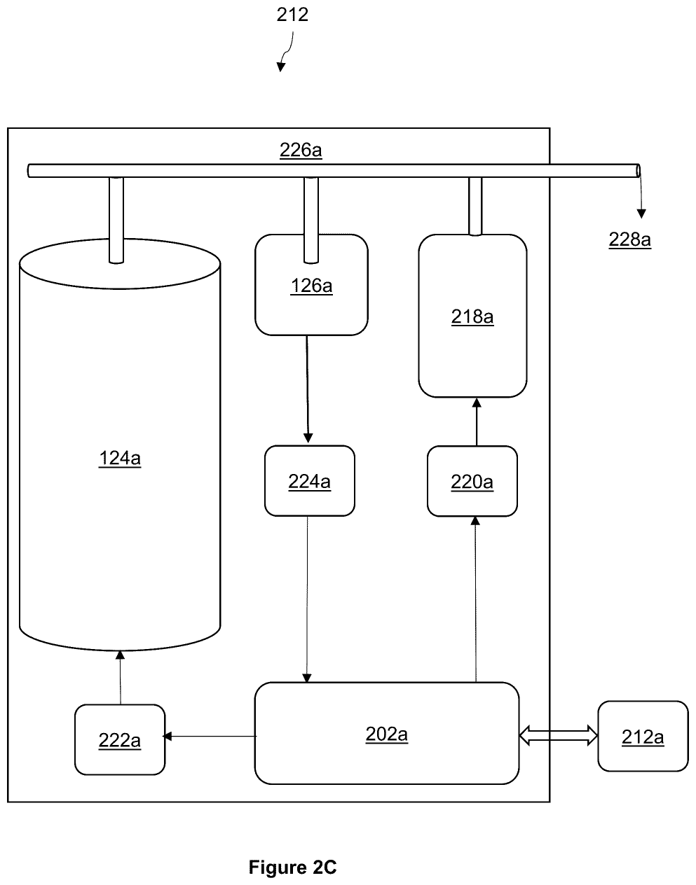

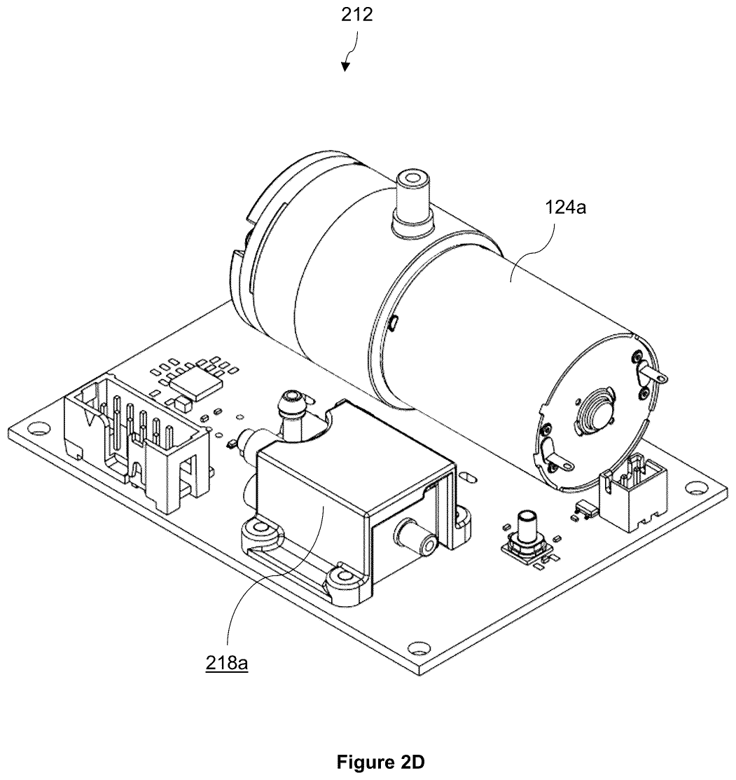

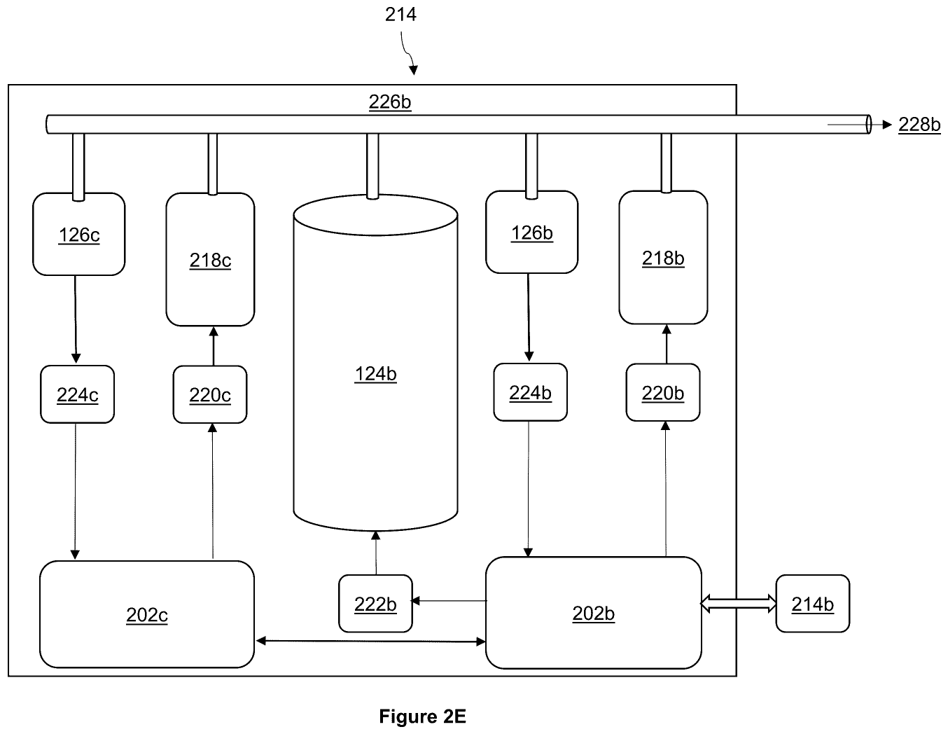

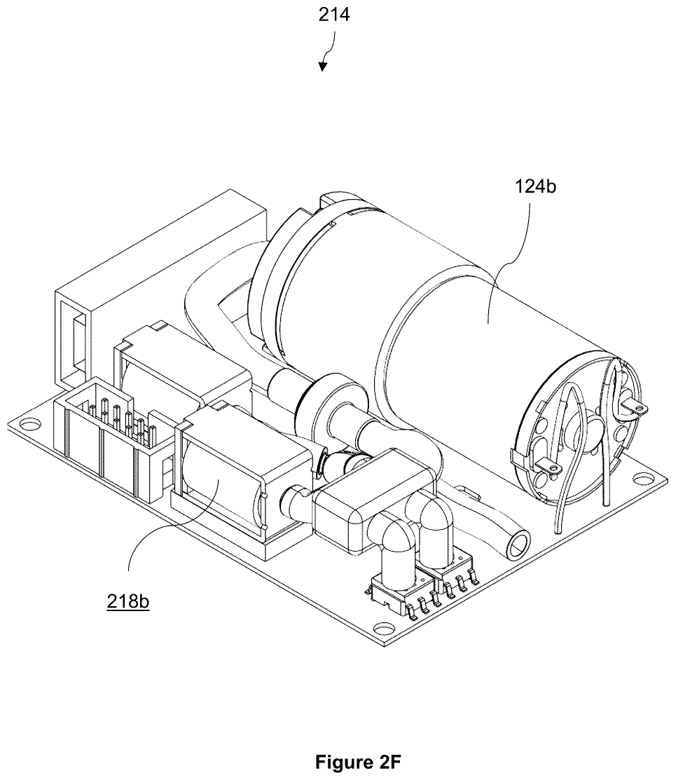

FIGS. 2A and 2B illustrates an exemplary block diagram representation of the control unit 134, in accordance with an embodiment of the present disclosure.

In an exemplary embodiment, the control unit 134, which is operatively connected to the one or more embedded sensors 132, the at least one linear actuation assembly 106, the elevation actuator 112, the displacement sensor 136, the dual-bladder cuff assembly 130, and the pneumatic subsystem 124. The control unit 134 coordinates the overall blood pressure measurement process, including adaptive positioning of the adaptive cuff positioning assembly 110, inflation and deflation of the fitment bladder 130a and the measurement bladder 130b, and processing of pressure data to determine the systolic pressure, the diastolic pressure, and the mean arterial pressure of the user.

The control unit 134 comprises a microcontroller 202 which serves as the central processing element. The microcontroller 202 executes computer readable instructions to process the posture data and the distance data received from the one or more embedded sensors 132 and the displacement sensor 136 and based on this posture data and the distance data, generates the one or more actuation commands to control the at least one linear actuation assembly 106, the elevation actuator 112, and the pivot assembly 114. The microcontroller 202 further controls inflation and deflation of the fitment bladder 130a and the measurement bladder 130b via the pneumatic subsystem 124 and acquires pressure signals from the one or more pressure sensors 126 associated with the dual-bladder cuff assembly 110.

The control unit 134 is powered through regulated supply stages comprising the low-dropout regulator (LDO) 5Volt (V) 204 and an LDO 3.3V 206. These LDOs (204 and 206) provide stable voltage levels for different components of the device 100, ensuring reliable operation of digital control circuits, analog sensing elements, and the communication module 128. For example, the microcontroller 202 and the communication module 128 may operate at 3.3V logic levels, while certain actuators or driver circuits may require 5V. The LDOs (204 and 206) are a type of linear voltage regulator configured to provide a stable output voltage even when the difference between the input voltage and the output voltage (dropout voltage) is very small, typically less than 0.3V. For example, the LDOs (204 and 206) may regulate a 5-volt input from a power adapter down to a stable 3.3-volt output required by the microcontroller or pressure sensors. The use of LDOs (204 and 206) ensures low noise, efficient operation, and protection of components from voltage fluctuations, thereby enhancing the accuracy and reliability of blood pressure measurement.

An integrated circuit RS232 driver 208 is included within the control unit 134 to enable serial data communication between the microcontroller 202 and the one or more end devices 216 through the communication module 128. This allows transmission of measured blood pressure data to the one or more end devices 216 such as, but not limited to, at least one of: a host computer, monitoring system, and remote healthcare platform for further processing or display. The integrated circuit RS232 driver 208 is provided in association with the communication module 128 and the control unit 134 to enable serial communication between the device 100 and the one or more end devices 216. The RS232 refers to the Recommended Standard 232, which is an established protocol for asynchronous serial data transmission commonly used in medical and industrial devices. The RS232 driver 208 converts the logic-level signals (for example, 0-3.3 volts or 0-5 volts) generated by the control unit 134 into the higher voltage levels (typically ±12 volts) required by RS232-compatible external devices such as the one or more end devices 216, and vice versa.

The control unit 134 also includes a power interface 210 to a power adapter, enabling connection to an external power supply. In one embodiment, the device 100 may operate using a mains adapter, and in another embodiment, the power interface 210 may support charging of an internal rechargeable battery. In another exemplary embodiment, the external power supply may be one of: a power adapter, or any other suitable type of power source. The internal rechargeable battery may be one of: a lithium-ion (Li-ion) battery, a lithium polymer (Li—Po) battery, a nickel-metal hydride (NiMH) battery, or any other medically approved rechargeable battery chemistry. In some embodiments, the power interface 210 may support universal serial bus (USB) charging, such as USB Type-C, enabling compatibility with standard charging accessories. In other embodiments, a dedicated charging dock or cradle may be used to recharge the internal rechargeable battery when the device 100 is not in use. The inclusion of the internal rechargeable battery ensures portability of the device 100, allowing it to be deployed in mobile healthcare units, rural settings, or home environments without constant reliance on mains power.

Additionally, a fitment bladder unit (FBU) interface 212a and a measurement bladder unit (MBU) interface 214a connects the control unit 134 to the fitment bladder unit (FBU) 212 and to the measurement bladder unit (MBU) 214, respectively. The fitment bladder unit (FBU) interface 212a and the measurement bladder unit (MBU) interface 214b are configured to send the one or more actuation commands and receive the pressure data from the fitment bladder 130a and the measurement bladder 130b. The FBU 212 is primarily responsible for inflating the fitment bladder 130a to grip the arm securely, while the MBU 214 inflates and deflates the measurement bladder 130b according to the predetermined pressure profile to obtain the oscillometric signals. The control unit 134 processes the pressure data transmitted from the MBU 214 and measures the blood pressure of the user.

In operation, the microcontroller 202 of the control unit 134 receives the posture data and the distance data, actuates the at least one linear actuation assembly 106, the elevation actuator 112 to align the adaptive cuff positioning assembly 110, initiates the inflation of the fitment bladder 130a through the FBU 212, and then controls the inflation and deflation of the measurement bladder 130b through the MBU 214. During the controlled deflation phase, the control unit 134 captures oscillometric signals via the one or more pressure sensors 126, applies the signal processing instructions, and determines the systolic pressure, the diastolic pressure, and the mean arterial pressure. The results are then communicated through the integrated circuit RS232 driver 208 and the communication module 128 to the one or more end devices 216.

Thus, the control unit 134 integrates power regulation, sensor interfacing, actuator control, bladder subsystem control, and external communication, thereby functioning as the central intelligence of the device 100 to enable automated and repeatable blood pressure measurement.

FIGS. 2C and 2D illustrates an exemplary block diagram representation of the fitment bladder unit (FBU) 212, in accordance with an embodiment of the present disclosure.

In an exemplary embodiment, the FBU 212 is operatively connected to the control unit 134 through the FBU interface 212a and is responsible for inflating and deflating the fitment bladder of the dual-bladder cuff assembly 130. The FBU 212 ensures that the fitment bladder 130a securely grips around the upper arm of the user prior to measuring the blood pressure, thereby stabilizing the adaptive cuff positioning assembly 110 and minimizing motion artifacts or the adaptive cuff positioning assembly displacement during operation.

The FBU 212 comprises a fitment bladder unit (FBU) diaphragm pump 124a of the one or more diaphragm pumps, which serves as the pneumatic subsystem 124 for generating airflow to inflate the fitment bladder 130a. The pneumatic subsystem 124 is driven by a fitment bladder unit (FBU) pump driver front end 222a under the one or more actuation commands of a fitment bladder unit (FBU) microcontroller 202a received from the control unit 134. The FBU pump driver front end 222a refers to the electronic circuitry operatively coupled to the pneumatic subsystem 124 and configured to drive one or more diaphragm pumps associated with the fitment bladder 130a. The FBU pump driver front end 222a functions as an electrical interface between the control unit 134 and the pneumatic subsystem 124, enabling precise and responsive inflation of the fitment bladder 130a to stabilize the adaptive cuff positioning assembly 110 around the upper arm of the user. The FBU diaphragm pump 124a is coupled through an FBU fluid conduit 226a of the distinct fluid conduits and an FBU tube interface 228a that physically links the pneumatic subsystem 124 output of the FBU 212 to the fitment bladder 130a housed within the cuff support structure 118.

The FBU 212 further comprises a fitment bladder unit (FBU) pressure sensor 126a of the one or more pressure sensor 126 positioned within the pneumatic pathway to continuously monitor the fitment bladder pressure data within the fitment bladder 130a. The FBU pressure sensor 126a generates an electrical signal proportional to the fitment bladder pressure data, which is processed by an FBU signal preprocessing stage 224a to condition and digitize the fitment bladder pressure data for use by the FBU microcontroller 202a. This real-time feedback ensures that the inflation of the fitment bladder 130a can be precisely controlled to achieve optimal gripping force around the user's arm without exceeding safe limits.

The pneumatic subsystem 124 further comprises a FBU solenoid valve 218a of the one or more solenoid valves connected to the pneumatic pathway and is actuated by a fitment bladder unit (FBU) valve driver front end 220a. The FBU solenoid valve 218a enables the controlled fluid communication from the fitment bladder 130a, thereby allowing rapid deflation via the one or more fail-safe vents when commanded by the control unit 134, in case of the fail-safe condition. This ensures that the fitment bladder 130a can be safely and quickly released from the arm of the user.

The FBU microcontroller 202a within the FBU 212 operates as a local controller for the pneumatic subsystem 124. The FBU microcontroller 202a receives the one or more actuation commands from the control unit 134 through the FBU interface 212a and coordinates the operation of the FBU diaphragm pump 124a, the FBU solenoid valve 218a, and the FBU pressure sensor 126a. Based on the distance data obtained from the displacement sensor 136 and the fitment bladder pressure data, the FBU microcontroller 202a modulates the FBU pump driver front end 222a to increase or decrease inflation rate and actuates the FBU solenoid valve 218a to release pressure as required.

In operation, when a measurement cycle is initiated, the control unit 134 signals the FBU 212 through the FBU interface 212a. The FBU diaphragm pump 124a inflates the fitment bladder 130a, the one or more pressure sensors 126 confirms the fitment bladder pressure data. The fitment bladder pressure data is monitored to verify secure gripping of the user's arm before inflation of the measurement bladder 130b begins. At the conclusion of the measurement cycle, or in the event of the safety condition such as overpressure or the power loss, the FBU solenoid valve 218a is automatically opened to the one or more fail-safe vents of the fitment bladder 130a, ensuring safe release of the dual-bladder cuff assembly 130 from the user's arm. Thus, the FBU 212 forms a critical part of the device 100, providing controlled inflation and deflation of the fitment bladder 130a to stabilize the dual-bladder cuff assembly 130 and ensure reproducible, safe, and reliable blood pressure measurements.

FIGS. 2E and 2F illustrates an exemplary block diagram representation of the measurement bladder unit (MBU) 214, in accordance with an embodiment of the present disclosure.

According to an exemplary embodiment, the MBU 214 is operatively connected to the control unit 134 and is responsible for controlled inflation and deflation of the measurement bladder 130b of the dual-bladder cuff assembly 130. The MBU 214 enables acquisition of the oscillometric signals during the controlled deflation phase, which are processed to determine the systolic pressure, the diastolic pressure, and the mean arterial pressure of the user.

The MBU 214 also comprises a measurement bladder unit (MBU) diaphragm pump 124b that serves as the pneumatic source for inflating the measurement bladder 130b. The MBU diaphragm pump 124b is driven by a measurement bladder unit (MBU) pump driver front end 222b, which is under the control of a measurement bladder unit (MBU) microcontroller unit 202b. The MBU pump driver front end 222b refers to the electronic circuitry operatively coupled to the pneumatic subsystem 124 and configured to drive one or more diaphragm pumps associated with the measurement bladder 130b. The MBU pump driver front end 222a functions as an electrical interface between the control unit 134 and the pneumatic subsystem 124, enabling precise and responsive inflation of the measurement bladder 130b to stabilize the adaptive cuff positioning assembly 110 around the upper arm of the user. The MBU diaphragm pump 124b is directed into the measurement bladder 130b through a measurement bladder unit (MBU) fluid conduit 226b of the distinct fluid conduits and a measurement bladder unit (MBU) tube interface 228b.

The pressure inside the measurement bladder 130b is continuously monitored by a measurement bladder unit (MBU) pressure sensor 126b of the one or more pressure sensors 126, which generates an analog signal proportional to the measurement bladder pressure data. The analog signal is processed through a measurement bladder unit (MBU) signal preprocessing stage 224b to remove noise and condition the analog signal for further processing by the MBU microcontroller unit 202b. The MBU microcontroller unit 202b allows precise control of inflation to a predetermined pressure (at least 20 mmHg above expected systolic pressure of the user) and a controlled deflation rate (typically 2-3 mmHg per second), as required for oscillometric blood pressure measurement.

A measurement bladder unit (MBU) solenoid valve 218b of the one or more solenoid valves is connected to the pneumatic pathway and controlled via a measurement bladder unit (MBU) valve driver front end 220b. The MBU solenoid valve 218b control the fluid communication (air) from the measurement bladder 130b, enabling stepwise or continuous deflation at the rate commanded by the MBU microcontroller unit 202b. The controlled deflation phase is critical for acquiring oscillometric waveforms from the MBU pressure sensor 126b.

To ensure safety and redundancy, the MBU 214 further incorporates an independent safety subsystem. The independent safety subsystem includes a safety pressure sensor 126c, a safety solenoid valve 218c, a safety pressure signal preprocessing stage 224c, a safety valve driver front end 220c, and a safety microcontroller unit 202c. The independent safety subsystem is configured to operate independently from a primary control loop and continuously monitor the measurement bladder pressure data. In the event of an overpressure condition, failure of the MBU solenoid valve 218b, or loss of communication with the MSU 214, the safety microcontroller unit 202c triggers the safety solenoid valve 218c to immediately release pressure from the measurement bladder 130b. The independent safety subsystem provides redundancy and ensures compliance with medical device safety standards for non-invasive blood pressure monitoring.

The MBU 214 communicates with the control unit 134 through the MBU interface 214b, enabling the transfer of the one or more actuation commands, measurement bladder pressure data, and fault signals. During a measurement cycle, the control unit 134 commands the MBU 214 to inflate the measurement bladder 130b to the target pressure, then initiates controlled deflation through the MBU solenoid valve 218b. The measurement bladder pressure data from the MBU pressure sensor 126b is transmitted to the control unit 134, where oscillometric instructions are executed to calculate blood pressure values. If the fault signals are detected, the control unit 134 triggers rapid venting through the MBU solenoid valve 218b or the safety subsystem can independently vent through the safety solenoid valve 218c.

In operation, the MBU 214 functions as a precision pneumatic and sensing module within the pneumatic subsystem 124 of the device 100. By providing controlled inflation and deflation of the measurement bladder 130b with integrated safety redundancies, the MBU 214 ensures accurate, repeatable, and safe determination of blood pressure.

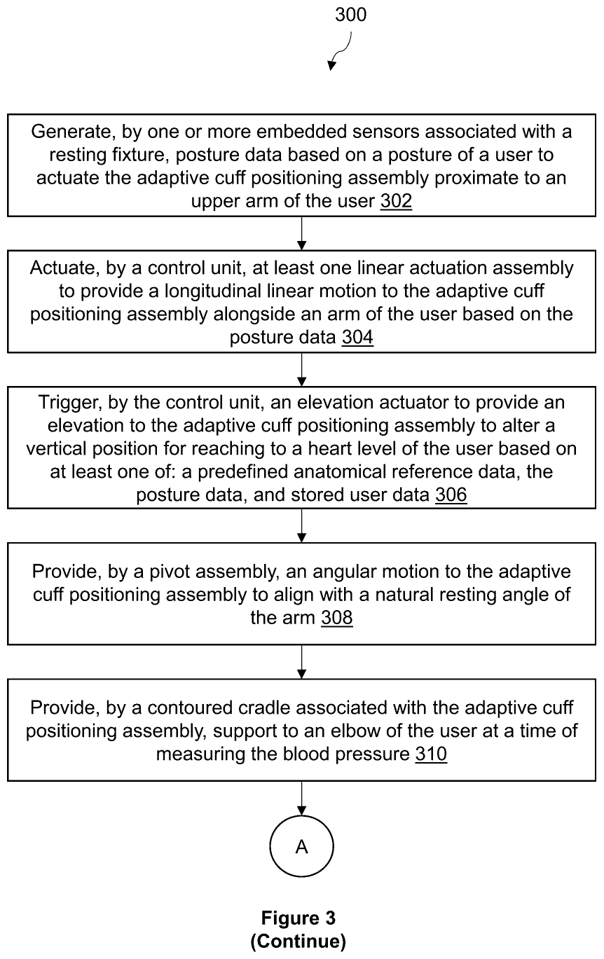

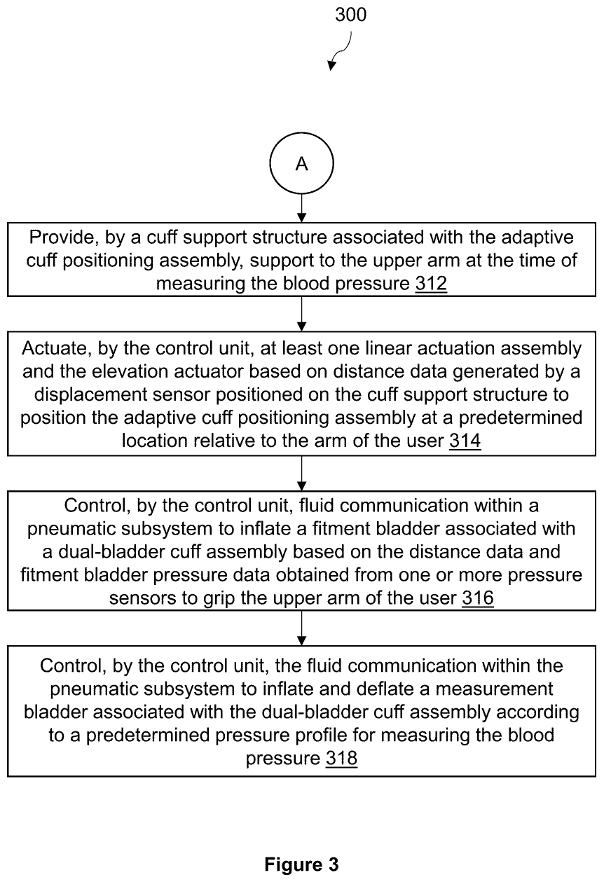

FIG. 3 illustrates an exemplary flow chart of a method 300 for measuring blood pressure using the adaptive cuff positioning assembly, in accordance with an embodiment of the present disclosure.

According to an exemplary embodiment of the present disclosure, the method 300 for measuring blood pressure using the adaptive cuff positioning assembly is disclosed. At step 302, the method 300 includes generating, by the one or more embedded sensors 132 associated with the resting fixture 102, the posture data based on the posture of the user to actuate the adaptive cuff positioning assembly 110 proximate to the upper arm of the user. The one or more embedded sensors 132 comprise at least one of: the load cell, the proximity sensor, and the optical sensor.

At step 304, the method 300 includes actuating, by the control unit 134, the at least one linear actuation assembly 106 to provide the longitudinal linear motion to the adaptive cuff positioning assembly alongside the arm of the user based on the posture data. The longitudinal linear motion is configured to translate the adaptive cuff positioning assembly 110 forward and backward relative to the arm of the user. This translation ensures that the adaptive cuff positioning assembly 110 can be positioned proximate to the upper arm of users having different arm lengths, while also enabling fine adjustment for alignment with the brachial artery. The at least one linear actuation assembly 106 comprising one of: the mechanical actuation unit, the hydraulic actuation unit, and the pneumatic actuation unit, operatively coupled to one of: the sliding rails, the guided linkage, the rack and pinion, the linear bearings and rails, the lead screw, and the ball screw.

At step 306, the method 300 includes triggering, by the control unit 134, the elevation actuator 112 to provide the elevation to the adaptive cuff positioning assembly 110 to alter the vertical position for reaching to the heart level of the user based on at least one of: the predefined anatomical reference data, the posture data, and the stored user data. The elevation actuator 112 may be implemented as one of, but not limited to, a telescopic actuator, a scissor lift actuator, a hydraulic cylinder, a pneumatic cylinder, a screw-driven actuator, and a linear motor.

At step 308, the method 300 includes providing, by the pivot assembly 114, the angular motion to the adaptive cuff positioning assembly to align with the natural resting angle of the arm. The pivot assembly 114 is configured as one of, but not limited to, a ball joint, a pin joint, a hinge joint, and the like.

At step 310, the method 300 includes providing, by the contoured cradle 116 associated with the adaptive cuff positioning assembly 110, support to the elbow of the user at the time of measuring the blood pressure. The contoured cradle 116 is formed with a recessed or cup-shaped geometry that conforms to the posterior aspect of the user's elbow joint, thereby preventing lateral or forward displacement of the arm during measurement.

At step 312, the method 300 includes providing, by the cuff support structure 118 associated with the adaptive cuff positioning assembly 110, support to the upper arm at the time of measuring the blood pressure. The cuff support structure 118 incorporates as a cylindrical-shaped design with an open segment, which is dimensioned to receive and support the medial and lateral aspects of the upper arm. This cylindrical geometry allows the dual-bladder cuff assembly 130 to be circumferentially positioned around the arm while maintaining a stable and centered orientation relative to the brachial artery.

At step 314, the method 300 includes actuating, by the control unit 134, the at least one linear actuation assembly 106 and the elevation actuator 112 based on the distance data generated by the displacement sensor 136 positioned on the cuff support structure 118 to position the adaptive cuff positioning assembly 110 at the predetermined location relative to the arm of the user. The displacement sensor 136 comprises at least one of: an ultrasonic sensor, an optical sensor, and a capacitive sensor, to determine a predetermined distance of at least one inch above an elbow crease of the user.

At step 316, the method 300 includes controlling, by the control unit 134, the fluid communication within the pneumatic subsystem 124 to inflate the fitment bladder 130a associated with the dual-bladder cuff assembly 130 based on the distance data and the fitment bladder pressure data obtained from the one or more pressure sensors 126 to grip the upper arm of the user. This grip stabilizes the arm within the cuff support structure 118 and ensures that the measurement bladder 130b is maintained in an anatomically correct orientation over the brachial artery.

At step 318, the method 300 includes controlling, by the control unit 134, the fluid communication within the pneumatic subsystem 124 to inflate and deflate the measurement bladder 130b associated with the dual-bladder cuff assembly 130 according to the predetermined pressure profile for measuring the blood pressure. The measurement bladder 130b is configured to inflate to a predetermined pressure at least 20 millimeters of mercury (mmHg) above a predictable systolic pressure of the user. For example, if the user's systolic pressure is expected to be 120 mmHg based on historical stored data or initial trial inflation, the measurement bladder 130b inflates to at least 140 mmHg. This ensures complete occlusion of the brachial artery, which is required for oscillometric measurement. The measurement bladder 130b is therefore configured to deflate in the controlled manner for obtaining the oscillometric signals that are analyzed to determine the systolic pressure, the diastolic pressure, and the mean arterial pressure of the user.

Numerous advantages of the present disclosure may be apparent from the discussion above. In accordance with the present disclosure, the device 100 enables measuring blood pressure of the user using the adaptive cuff positioning assembly 110 without manual intervention, thereby overcoming limitations of conventional blood pressure monitors that rely on operator skill and manual cuff placement. The integration of the one or more embedded sensors 132 with the control unit 134 allows automatic detection of the posture and the arm position of the user, ensuring that the adaptive cuff positioning assembly 110 is consistently aligned with the upper arm at the predetermined anatomical reference location above the elbow crease.

The inclusion of the at least one linear actuation assembly 106, the elevation actuator 112, and the pivot assembly 114 enables precise adjustment of the adaptive cuff positioning assembly 110 in longitudinal, vertical, and angular directions, thereby aligning the cuff centerline with the heart level and the natural resting angle of the arm. This automated positioning reduces user-to-user variability, enhances comfort, and minimizes measurement error associated with improper cuff alignment.

Further, the dual-bladder cuff assembly 130, comprising the fitment bladder 130a and the measurement bladder 130b, ensures both stability and accuracy during measurement. The fitment bladder 130a secures the cuff around the arm, while the measurement bladder 130b inflates and deflates according to a controlled pressure profile to obtain the oscillometric signals for determining the systolic pressure, the diastolic pressure, and the mean arterial pressure. Independent fluid conduits, controlled by the pneumatic subsystem 124 and monitored by the one or more pressure sensors 126, ensure safe and reliable bladder operation.

The device 100 further provides patient safety through features such as the one or more fail-safe vents, overpressure monitoring, and rapid deflation control, preventing excessive pressure on the user's arm during operation or in case of system malfunction. Additionally, the ability of the device 100 to initiate measurement automatically upon detection of posture data minimizes the need for manual input, reducing operator dependency and improving ease of use in clinical, home, or telemedicine environments.

Accordingly, the device 100 offers improved reliability, repeatability, and user comfort in non-invasive blood pressure measurement, thereby addressing the drawbacks of existing technologies and ensuring that accurate results can be obtained across a wide range of users and operating conditions.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

What is claimed is:

1. A device for measuring blood pressure using an adaptive cuff positioning assembly, the device comprising:
a resting fixture configured with one or more embedded sensors to detect posture of a user for generating posture data to actuate the adaptive cuff positioning assembly proximate to an upper arm of the user; and
at least one armrest operatively coupled to the resting fixture, configured to hold the adaptive cuff positioning assembly alongside the resting fixture;
at least one linear actuation assembly operatively coupled on the at least one armrest, configured to provide a longitudinal linear motion to the adaptive cuff positioning assembly alongside an arm of the user based on the posture data;
an enclosure pivotally coupled to the at least one linear actuation assembly using an elevation actuator and a pivot assembly, configured to provide support to a forearm of the user,
the elevation actuator configured to provide an elevation to the adaptive cuff positioning assembly to alter a vertical position for reaching to a heart level of the user based on at least one of: predefined anatomical reference data, the posture data, and stored user data; and
the pivot assembly configured to provide an angular motion to the adaptive cuff positioning assembly to align with a natural resting angle of the arm;
the adaptive cuff positioning assembly operatively coupled to the enclosure, the adaptive cuff positioning assembly comprises:
a contoured cradle operatively positioned on the enclosure, configured to provide support to an elbow of the user at a time of measuring the blood pressure;
a cuff support structure operatively connected to the contoured cradle, configured to provide support to the upper arm at the time of measuring the blood pressure;
a displacement sensor operatively positioned on the cuff support structure, configured to generate distance data used to position the adaptive cuff positioning assembly at a predetermined location relative to the arm of the user;
a dual-bladder cuff assembly operatively positioned inside the cuff support structure, the dual-bladder cuff assembly comprises:
a fitment bladder configured to inflate by a pneumatic subsystem based on the distance data for gripping around the upper arm of the user; and
a measurement bladder configured to inflate and deflate by the pneumatic subsystem according to a predetermined pressure profile for measuring the blood pressure; and
a control unit operatively associated with the enclosure and operatively connected to the one or more embedded sensors, the at least one linear actuation assembly, the elevation actuator, the displacement sensor, the dual-bladder cuff assembly, and one or more pressure sensors,
the control unit configured to process the posture data and the distance data to generate one or more actuation commands for:
actuating the at least one linear actuation assembly in the longitudinal linear motion;
triggering the elevation actuator to provide the elevation to the adaptive cuff positioning assembly; and
controlling fluid communication within the pneumatic subsystem to inflate and deflate the fitment bladder and the measurement bladder, based on fitment bladder pressure data and measurement bladder pressure data obtained from the one or more pressure sensors, to measure the blood pressure.

2. The device of claim 1, wherein the one or more embedded sensors comprise at least one of:
a load cell configured to determine weight of the user on the resting fixture for generating the posture data;
a proximity sensor configured to determine distance between the adaptive cuff positioning assembly and the arm of the user for generating the posture data; and
an optical sensor configured to detect the posture of the user for determining the heart level of the user for generating the posture data.

3. The device of claim 1, wherein the longitudinal linear motion provided by the at least one linear actuation assembly is configured to translate the adaptive cuff positioning assembly forward and backward relative to the arm of the user; and
the at least one linear actuation assembly comprising one of: a mechanical actuation unit, a hydraulic actuation unit, and a pneumatic actuation unit, operatively coupled to one of: sliding rails, a guided linkage, rack and pinion, linear bearings and rails, a lead screw, and a ball screw.

4. The device of claim 1, wherein the elevation actuator is configured as one of: a telescopic actuator, a scissor lift actuator, a hydraulic cylinder, a pneumatic cylinder, a screw-driven actuator, and a linear motor, to provide the elevation to the adaptive cuff positioning assembly.

5. The device of claim 1, wherein the pivot assembly is configured as one of: a ball joint, a pin joint, and a hinge joint, to provide the angular motion to the adaptive cuff positioning assembly.

6. The device of claim 1, wherein the displacement sensor comprises at least one of: an ultrasonic sensor, an optical sensor, and a capacitive sensor, to determine a predetermined distance of at least one inch above an elbow crease of the user.

7. The device of claim 1, wherein the fitment bladder and the measurement bladder are independently connected to the pneumatic subsystem through distinct fluid conduits.

8. The device of claim 1, wherein the measurement bladder is configured to inflate to a predetermined pressure at least 20 millimeters of mercury (mmHg) above a predictable systolic pressure of the user and to deflate according to a controlled deflation rate between 2 mmHg per second and 3 mmHg per second, defined by the control unit based on the predetermined pressure profile,
the measurement bladder configured to deflate for obtaining oscillometric signals for determining systolic pressure, diastolic pressure, and mean arterial pressure of the user.

9. The device of claim 1, wherein the pneumatic subsystem comprises one or more diaphragm pumps, one or more solenoid valves, and one or more fail-safe vents, to control inflation and deflation of the fitment bladder and the measurement bladder.

10. The device of claim 1, wherein the control unit is configured to monitor the fitment bladder pressure data to verify optimal gripping of the upper arm of the user before initiating inflation of the measurement bladder.

11. The device of claim 1, wherein the control unit is further configured to trigger rapid deflation of the fitment bladder and the measurement bladder through the one or more fail-safe vents based on one of: the fitment bladder pressure data, the measurement bladder pressure data, and power failure conditions.

12. The device of claim 1, wherein the control unit configured to initiate measuring the blood pressure upon obtaining the posture data from the one or more embedded sensors.

13. A method for measuring blood pressure using an adaptive cuff positioning assembly, the method comprising:
generating, by one or more embedded sensors associated with a resting fixture, posture data based on a posture of a user to actuate the adaptive cuff positioning assembly proximate to an upper arm of the user;
actuating, by a control unit, at least one linear actuation assembly to provide a longitudinal linear motion to the adaptive cuff positioning assembly alongside an arm of the user based on the posture data;
triggering, by the control unit, an elevation actuator to provide an elevation to the adaptive cuff positioning assembly to alter a vertical position for reaching to a heart level of the user based on at least one of: a predefined anatomical reference data, the posture data, and stored user data;
providing, by a pivot assembly, an angular motion to the adaptive cuff positioning assembly to align with a natural resting angle of the arm;
providing, by a contoured cradle associated with the adaptive cuff positioning assembly, support to an elbow of the user at a time of measuring the blood pressure;
providing, by a cuff support structure associated with the adaptive cuff positioning assembly, support to the upper arm at the time of measuring the blood pressure;
actuating, by the control unit, at least one linear actuation assembly and the elevation actuator based on distance data generated by a displacement sensor positioned on the cuff support structure to position the adaptive cuff positioning assembly at a predetermined location relative to the arm of the user;
controlling, by the control unit, fluid communication within a pneumatic subsystem to inflate a fitment bladder associated with a dual-bladder cuff assembly based on the distance data and fitment bladder pressure data obtained from one or more pressure sensors to grip the upper arm of the user; and
controlling, by the control unit, the fluid communication within the pneumatic subsystem to inflate and deflate a measurement bladder associated with the dual-bladder cuff assembly according to a predetermined pressure profile for measuring the blood pressure.

14. The method of claim 13, wherein the one or more embedded sensors comprise at least one of:
a load cell configured to determine weight of the user on the resting fixture for generating the posture data;
a proximity sensor configured to determine distance between the adaptive cuff positioning assembly and the arm of the user for generating the posture data; and
an optical sensor configured to detect the posture of the user for determining the heart level of the user for generating the posture data.

15. The method of claim 13, wherein the displacement sensor comprises at least one of:
an ultrasonic sensor, an optical sensor, and a capacitive sensor, to determine a predetermined distance of at least one inch above an elbow crease of the user.

16. The method of claim 13, wherein the fitment bladder and the measurement bladder are independently connected to the pneumatic subsystem through distinct fluid conduits.

17. The method of claim 13, wherein
inflating the measurement bladder to a predetermined pressure at least 20 millimeters of mercury (mmHg) above a predictable systolic pressure of the user; and
deflating the measurement bladder according to a controlled deflation rate between 2 mmHg per second and 3 mmHg per second, to obtain oscillometric signals for determining systolic pressure, diastolic pressure, and mean arterial pressure of the user.

18. The method of claim 13, wherein the pneumatic subsystem comprises one or more diaphragm pumps one or more solenoid valves, and one or more fail-safe vents, to control inflation and deflation of the fitment bladder and the measurement bladder.

19. The method of claim 13, further comprising:
monitoring, by the control unit, the fitment bladder pressure data to verify optimal gripping of the upper arm of the user before initiating inflation of the measurement bladder.

20. The method of claim 13, further comprising:
triggering, by the control unit, rapid deflation of the fitment bladder and the measurement bladder through the one or more fail-safe vents based on one of: the fitment bladder pressure data, measurement bladder pressure data, and power failure condition.
